(19) United States
(12) Patent Application Publication
(10) Pub. No.: US 2002/0031562 A1
(43) Pub. Date: Mar. 14, 2002

(54) Recovery of gaultherin from plants

(76) Inventors: David M. Ribnicky, Monmouth Junction, NJ (US); Alexander A. Poulev, Highland Park, NJ (US); Ilya Raskin, Manalapan, NJ (US)

Correspondence Address: MARSHALL, O'TOOLE, GERSTEIN, MURRAY & BORUN, 6300 SEARS TOWER, 233 SOUTH WACKER DRIVE, CHICAGO, IL, 60606-6402, US

(21) Appl. No.: 09/887,766

(22) Filed: Jun. 21, 2001

Related U.S. Application Data
(63) Continuation-in-part of application No. 09/258,597, filed on Feb. 26, 1999.

(51) Int. Cl. A61K 35/78; C07H 13/02

(52) U.S. Cl. 424/769; 536/119

(57) ABSTRACT

The present invention provides methods for obtaining gaultherin, a natural salicylate derivative, from plant tissue. The methods involve preventing the hydrolysis of the gaultherin in the plant tissue by disrupting the plant tissue under solvent conditions lacking a drying agent. The invention further provides various forms of plant-derived gaultherin, including alcohol extracts, aqueous solutions and dried preparations, all suitable for use as a natural aspirin substitute.

This application is a continuation-in-part of U.S. patent application Ser. No. 09/258,597, filed Feb. 26, 1999.

### FIELD OF THE INVENTION

This invention relates to the field of pharmaceutically active compounds obtained from natural sources. In particular, the invention provides a salicylate derivative, gaultherin, isolated from plant sources, particularly Gaultheria procumbens, as well as methods for obtaining high yields of the compound from the plant source.

### BACKGROUND OF THE INVENTION

Various scientific articles are referred to in parentheses throughout the specification, and complete citations are listed at the end of the specification. These articles are incorporated by reference herein to describe the state of the art to which this invention pertains.

The medical benefits of plant salicylates have been enjoyed by people for centuries. Today, aspirin (acetylsalicylic acid) is probably the most widely used drug in the world because of its antipyretic, anti-inflammatory and analgesic properties.

Salicylic acid was first isolated in 1839 from the flower buds of the herb called Filipendula ulmaria or Spiraea ulmaria. The benefits of plant-derived salicylates prompted intensive research, which led to the commercial production of synthetic acetylsalicylic acid (aspirin) in 1899.

The development of the acetylated form of salicylate was prompted by the need for a form of the drug that would not cause the gastrointestinal trouble associated with the use of salicylic acid. Indeed, acetylsalicylic acid has been shown to have fewer side effects than salicylic acid, but can promote similar problems.

Most of the pharmacological activity of acetylsalicylic acid is due to the production of salicylic acid. Some noteworthy activities of salicylic acid include general anti-inflammatory properties, increased fibrinolysis, inhibition of glycosaminoglycan synthesis, inhibition of the lipoxygenase pathway, reduction of T-cell adhesion, free radical scavenging, inhibition of prostaglandin biosynthesis and some anti-carcinogenesis activities.

As mentioned, acetylsalicylic acid was synthesized as a derivative of salicylic acid with fewer side effects. Efforts have been made to derivatize salicylic acid and aspirin in various ways to further mitigate gastric irritation. The general strategy for reducing gastric upset has been to chemically derivatize the salicylate molecule to delay the release of free salicylic acid until after it has passed from the stomach. Such derivatized forms, which include substituents such as sugars, phenolics and triglycerides, have been shown to be efficacious.

Plant species that contain high concentrations of salicylic acid naturally produce such derivatives as safe storage forms. Wintergreen, Gaultheria procumbens, contains a very high concentration of salicylate derivatives, reaching concentrations exceeding 10 mg per gram fresh weight of tissue. This concentration is over 20-fold greater than that found in Filipendula, from which salicylic acid was first isolated. Reports typifying the work done in Filipendula are found, e.g., in the abstracts of Barnaulov et al., Rastit. Resur. 13(4), 661-9 (1977) and Yeo et al., Saengyak Hakhoechi 23 (3), 121-5 (1992).

The majority of salicylate present in G. procumbens is found in a form called gaultherin, which consists of methyl salicylate conjugated to the disaccharide, primeverose. Methyl salicylate, also known as oil of wintergreen, is responsible for the smell and taste of wintergreen. When plant tissues are disrupted, the endogenous gaultherin is enzymatically hydrolyzed and methyl salicylate is released. This process presumably occurs as a protective mechanism for the plant.

In 1844, Proctor defined gaultherin as a conjugate of methyl salicylate with glucose but claimed that it did not exist within the plant for which it was named. Interest in such conjugates did not recur until nearly 60 years later, when a series of articles was published in France. The authors of these articles (Bridel et al., 1995; Bridel and Guignard, 1925 a,b; Birdel and Guignard, 1923a,b; Goris et al., 1919) described the sugars of these conjugates and defined monotropidoside as a conjugate of methyl salicylate with primeverose, which is a disaccharide of xylose and glucose. These studies were performed with various species, leading to the occurrence of excessive terminology, most of which seemed, in retrospect, to describe the same conjugate or enzyme activity.

Gaultheria procumbens was not examined until 1928, when it was determined that monotropidoside was the same as gaultherin and that gaultherin could only be extracted from Gaultheria with boiling water and calcium carbonate, followed by a series of solvent extractions, including 95% alcohol distillation, extraction with boiling hydrated acetic ether and addition of 95% alcohol (Bridel and Gillon, 1928) which gave a final yield of 4 g/kg fresh weight plant material. These combined observations described gaultherin as a conjugate of methyl salicylate with a disaccharide of xylose and glucose. These references also described an enzymatic activity leading to the hydrolysis of this conjugate and designated it as gaultherase. Those terms have been perpetuated by the literature but no current investigations have been performed on either the conjugate or the enzyme (which is defined as an activity only). The previous work was summarized in 1931 (Robertson and Waters, 1931) along with a description of the synthesis of gaultherin. Any current literature which includes the terms gaultherin or gaultherase appear to refer to these original sources.

From the foregoing, it can be seen that gaultherin possesses all the features of an ideal natural analog of aspirin. Gaultherin is found in plant tissues at high concentrations and is an extensively derivatized form of salicylate, which should result in minimal gastric side effects. Moreover, although methyl salicylate can be toxic when ingested at concentrations used for topical application, this ester has been shown to have decreased ulcerogenic activity as well. Accordingly, gaultherin should prove to be an effective natural substitute for synthetic aspirin, to be taken on a daily basis for general cardio-pulmonary benefit, or on an as-needed basis as a pain reliever and anti-inflammatory agent.

Unfortunately, an effective method for obtaining useful quantities of gaultherin from natural plant sources is not currently available. The difficulty in obtaining gaultherin from plant tissue resides in the fact that, upon disruption of the tissue, the molecule is immediately hydrolyzed to its individual components, methyl salicylate and a disaccharide. The hydrolysis is believed to be catalyzed by an enzyme referred to as gaultherase (see Robertson & Waters (1931) J. Am. Chem. Soc. pp. 1881-1889, and references cited therein). Regardless of the means by which the hydrolysis occurs, it appears to be immediate and essentially complete, inasmuch as it has led some investigators to conclude that certain plants, most notably Gaultheria procumbens, do not contain gaultherin, when indeed they do (See Proctor (1844), Am. J. Pharmacol., Vol IX, No. IV., pp. 22 and 242-250).

U.S. Pat. No. 5,176,913, issued Jan. 5, 1993, to Honerlagen et al. (Honerlagen) describes a method for preparing a partial extract containing the volatile-in-steam components and further lipophilic components of various plants. Among many plant species, Gaultheria procumbens L. is disclosed as a plant from which an extract can be obtained. Honerlagen teaches a method wherein a drying agent is brought into contact with a crude extract to reduce or eliminate the water content of the extract. Honerlagen isolates lipophilic plant compounds, including both volatile-in-steam and non-volatile-in-steam compounds, to recover compounds generally not soluble in water. However, there is no recognition or appreciation in Honerlagen of a method that allows the separation and recovery of gaultherin from any accompanying gaultherase activity to protect gaultherin from hydrolysis.

Thus, there remains a need in the art for a method for recovering gaultherin from plants by extraction under conditions that minimize or eliminate gaultherin degradation or destruction, such as hydrolysis due to a gaultherase activity.

### SUMMARY OF THE INVENTION

The present invention provides a solution to the aforementioned problem by providing a convenient method of obtaining gaultherin, as opposed to its products of hydrolysis, from natural plant sources, particularly Gaultheria procumbens. The present invention also provides plant extracts containing gaultherin and purified gaultherin obtained from such plant sources, for use as a natural aspirin analog exhibiting minimal gastric side effects.

According to one aspect of the present invention, a plant extract containing gaultherin is provided. Preferably, the extract is obtained from Gaultheria procumbens, and preferably, a yield of at least 5 mg gaultherin per gram fresh weight plant material is obtained. The high yield of gaultherin is achieved by extracting fresh or frozen plants or plant parts, such as those obtained from Gaultheria procumbens, in a solvent lacking a drying agent (i.e., tragacanth, gelatin, a water-free sodium sulfate, a water-free magnesium sulfate, a water-free calcium chloride, a molecular sieve, or combinations thereof, as examples of compounds that bind, absorb, adsorb, or capture water molecules) for purposes of removing or reducing the water content in a mixture or composition. In particular, the gaultherin is recovered in a solvent that has sufficient polarity to retain gaultherin while reducing the level of a gaultherase activity. Preferably, no more than 20% of the gaultherase activity is recovered (relative to the activity recovered using a suitably buffered water solvent under otherwise comparable extraction conditions); more preferably, no more than 5% of the gaultherase activity is recovered and most preferably, no more than 1% of the gaultherase activity is recovered. The methods of the invention thus provide improved yields of gaultherin in plant extracts, which are useful as nutraceutical compositions and as therapeutics.

Preferably, the extract comprises at least 10 mg gaultherin per gram fresh weight (gfw) plant material, 5 more preferably 15 mg/gfw, even more preferably 20 mg/gfw, and most preferably it comprises at least 25 mg gaultherin per gram fresh weight plant material. In a preferred embodiment, the extract is an alcoholic extract wherein the alcohol is selected from the group consisting 10 of methanol, ethanol and isopropanol. In a more preferred embodiment, gaultherin is present in an ethanolic extract and can be ingested or applied, or incorporated into a nutraceutical or pharmaceutical composition for ingestion or application to a mammal such as a human. In another preferred embodiment, a dried or powdered preparation of gaultherin obtained from the alcoholic or extract or an aqueous resuspension thereof is provided.

According to another aspect of the invention, a 20 method is provided for obtaining gaultherin from plants. Preferably, the plant is Gaultheria procumbens, and, preferably, the method yields gaultherin in an amount of at least 5 mg, preferably 10 mg, more preferably 15 mg, even more preferably 20 mg, and most preferably 25 mg per 25 gram fresh weight plant material. The method comprises: (a) providing fresh or fresh-frozen plant material; and (b) disrupting the tissue under solvent conditions lacking a drying agent that reduce the likelihood of hydrolysis of gaultherin, thereby producing an extract of the tissue. Suitable solvents will have sufficient polarity to retain gaultherin, while reducing the recovery yield of any gaultherase activity. Preferably, the tissue is disrupted in the presence of an alcohol solvent to produce an alcoholic extract. In a preferred embodiment, the alcohol is ethanol.

In related embodiments, the method may further comprise removing particulate plant material from the extract. In addition, the method may be extended to provide a powdered (i.e., solid) preparation of a gaultherin-containing composition prepared by drying the extract. Where the extract is dried, it is preferred that the extract is exposed to a compound that removes all solvent components, including, for example, aqueous, non-polar, and polar fluids.

In yet another embodiment, the method further comprises adding water to the dried extract, thereby producing an aqueous solution of gaultherin. The aqueous solutions, another aspect of the invention, also may be dried to produce a more purified solid form of gaultherin.

The extracts, aqueous solution and solid residue containing gaultherin described herein can be used to advantage as a natural aspirin substitute which is less gastrically irritating than aspirin. Other features and advantages of the present invention will be better understood by reference to the drawings, detailed description and examples that follow.

### BRIEF DESCRIPTION OF THE DRAWINGS

FIG. 1. LC-MS profiles of wintergreen extracts showing comparative stability of gaultherin in methanol (upper panel) and water (lower panel).

FIG. 2. HPLC profiles of wintergreen extracts showing comparative stability of gaultherin in methanol (upper panel) and water (lower panel).

FIG. 3A and 3B. Liquid chromatography/mass spectrometry of bound forms of salicylate from wintergreen (Gaultheria procumbens). FIG. 3A: LC-MS profile of gaultherin, showing a major peak (P2) identified as gaultherin and a minor peak (P1) presumed to be a gaultherin variant. FIG. 3B: Mass spectrograms of P1 and P2.

### DETAILED DESCRIPTION OF THE INVENTION

Prior to the present invention, no method had been available for isolating significant amounts of gaultherin from plant tissue, due to the rapid degradation of the molecule into its components, methyl salicylate and primeverose, upon disruption of the plant tissue. As a result, this useful and advantageous form of salicylate has gone unexploited.

In accordance with the present invention, it has now been discovered that gaultherin can be isolated intact from plant tissue, using a simple process that inhibits the aforementioned hydrolytic breakdown of the molecule. This process is most advantageously applied to the wintergreen plant, which, surprisingly in view of reports to the contrary, has now been discovered to have a very high gaultherin content as compared to other selected plant species. The description below exemplifies wintergreen as the plant of choice for obtaining significant quantities of gaultherin. However, it will be appreciated by persons skilled in the art that the same methods could be applied to any plant species containing gaultherin or a similar salicylate derivative, with an expectation of obtaining intact gaultherin or similar derivative in high proportion to whatever amount is contained within that species. The rapid hydrolysis of gaultherin to form methyl salicylate and the disaccharide, primeverose, has been attributed to an enzymatic activity referred to as “gaultherase”. Gaultherase has never been isolated, so it is as yet unproven as to whether the hydrolysis of gaultherin is catalyzed by an enzyme. Regardless of the mechanism by which hydrolysis occurs, however, it is clear that disruption of the cells of gaultherin-containing plant tissue results in immediate hydrolysis of gaultherin. The present inventors have discovered that this hydrolytic activity, whatever its cause, can be inhibited by disrupting the plant tissue in the presence of alcohol. It is believed that the alcohol inhibits the enzymatic activity of gaultherase, but other mechanisms may also play a role. It is also expected that other solvents, as listed below, or boiling water, will act in a manner similar to alcohol, to inhibit hydrolysis of gaultherin upon disruption of the plant cells.

Thus, the inventors have developed an extraction method for obtaining high yields of gaultherin or similar salicylate conjugates (e.g., other forms of sugar-conjugated methyl salicylate) from plant sources, particularly Gaultheria procumbens. In its most basic form, the method comprises the following steps: (1) provide fresh or fresh-frozen plant material; (2) optionally, freeze the tissue in liquid nitrogen; and (3) grind or otherwise disrupt the tissue in a solution containing a sufficient amount of alcohol (e.g., ethanol) or other solvent lacking a drying agent. The frozen, macerated tissue can be stored frozen for further processing. The extract may be further processed in the following steps: (4) remove solid matter from the alcoholic extract; (5) subjecting the alcoholic extract to an agent that removes solvent components to produce a solid (powdered) gaultherin-containing residue; (6) re-suspend the resulting alcoholic extract in an aqueous solution; and (7) after removing any water insoluble material, repeating step (5) to form a more purified form of gaultherin.

Using the aforementioned extraction procedure on Gaultheria procumben, gaultherin has been obtained from above-ground plant parts in the amount of 1-26 mg per gram fresh weight tissue. The sections below describe each step of the extraction process in greater detail.

As mentioned above, the plant species chosen for obtaining gaultherin plays a very important role in how much gaultherin can be obtained from the plant source. Gaultheria procumbens contains a very high concentration of salicylate, mainly in the form of gaultherin (see Example 1). However, other plant species also contain significant amounts of bound salicylate, and could be used instead of Gaultheria procumbens as a plant source of derivatized salicylate such as gaultherin. These include, but are not limited to, various members of the thyme family, such as English thyme, French thyme and lemon thyme. However, wintergreen far exceeds any of these plant species in bound salicylate content.

The cultivar of wintergreen plant, as well as growth conditions of the plants, also affect the gaultherin content of the plant. For instance, though cultivated wintergreen is not categorized into varieties, the type of plant cultivated on the west coast of the United States has been found to have a higher salicylate content than does the wintergreen cultivated on the east coast (see Table II of Example 1). Additionally, the conditions under which the plants are grown also affect their gaultherin content. In particular, the inventors have noted that plants subjected to stress conditions, such as heat stress, dehydration or exposure to chemical elicitors, have a higher bound salicylate content than do plants not subjected to such conditions. Accordingly, one modification of the extraction method of the invention to maximize gaultherin yield is to stress the plants prior to harvesting them for the extraction.

The salicylate content in wintergreen plants also varies somewhat with the tissue type. As shown in Table IV of Example 1, flowers tend to have a higher salicylate content than do leaves, stems and berries. Thus, another modification of the extraction method of the invention is to use only wintergreen flowers as the starting plant material. However, a more feasible alternative is to use all above-ground plant parts, inasmuch as the leaves, stems and berries of wintergreen do contain significant concentrations of salicylate, and it is much less labor intensive simply to harvest the entire plant.

In a preferred embodiment, fresh plant tissue is quick-frozen in liquid nitrogen, then ground or otherwise macerated (e.g., using a Polytron or a Waring blender) in alcohol. For this purpose, it is preferable to use an alcohol preparation containing 70-95% alcohol. However, preparations containing as little as 30% alcohol have been found reasonably effective for extracting gaultherin. Alcohols preferred for practice of the invention include lower alkyl alcohols, such as methanol, ethanol or isopropanol. A particularly preferred alcohol for the extraction step of the invention is ethanol. A benefit of incorporating an ethanolic solvent in the extraction step of the invention is that gaultherin is surprisingly stable in an ethanolic solvent, which provides the advantage of an increased gaultherin yield or recovery. In addition, an ethanolic solvent is compatible with an ingestible nutraceutical product and, therefore, is suitable for incorporation into a pill, capsule, tablet or other ingestible form known in the art. It will be appreciated that any alcohol which inhibits the hydrolysis of gaultherin in the extraction process is contemplated for use in the present invention. Other alcohols suitable for use in the invention include, but are not limited to, n-propyl alcohol or any form of butanol, pentanol or hexanol, among others. In addition, the initial extraction may be performed with other solvents, including, but not limited to, methylene chloride, acetonitrile, acetone and chloroform. The initial extraction also may be performed with very hot water, preferably at the boiling point, which may, but need not, act by providing a suitably polar solvent for recovery of gaultherin while providing unsuitable conditions for recovery of an active form of a gaultherase activity.

The extract of plant material preferably is separated from the solids in the extract, e.g., by filtration, centrifugation, or any commonly known method. The gaultherin content of the extract may then be tested by known methods, or preferably, using any of the methods developed in accordance with the present invention, which are set forth in Example I. These include, but are not limited to, high performance liquid chromatography or liquid chromatography/mass spectrometry, or hydrolysis in acid followed by analysis by gas chromatography/mass spectrometry, as combined with stable isotope dilution analysis.

A solid residue containing gaultherin may be prepared by removing the solvent from the alcoholic extract. Preferably, the residue contains about 12-18% (by weight) gaultherin. In a preferred embodiment of the invention, the alcohol extract is dried by removing the solvent therefrom, then re-dissolved in water or buffer.

Water-insoluble materials are removed, e.g. by centrifugation, to prepare an aqueous solution containing the gaultherin. In a further preferred embodiment, the aqueous solution is again reduced to dryness to produce a residue highly enriched in gaultherin.

Any of the preparations described above, i.e., the alcoholic or other solvent extract, aqueous solution or dried preparations, may be used as a natural alternative to acetylsalicylic acid, or “aspirin.” The advantages of using gaultherin as an aspirin substitute include the fact that the compound is a “natural” compound, isolated from a plant source using a simple extraction process, as well as the fact that gaultherin is a highly derivatized salicylate that should cause less gastric irritation than does aspirin.

The alcoholic extract of gaultherin may be applied topically, or if prepared with an ingestible alcohol, may be administered orally or intranasally. Similarly, the aqueous solution of gaultherin may be administered orally or intranasally, or by any other means known for administration of aqueous solutions of acetylsalicylic acid. The dried gaultherin preparations can be tabletted or encapsulated or otherwise formulated for oral administration (e.g., in a gum or candy). For any of the liquid or solid formulations, the gaultherin preferably is administered as a dosage unit. The term “dosage unit” refers to a physically discrete unit of the preparation appropriate for a patient undergoing treatment or using the compound for prophylactic purposes. Each dosage unit contains a quantity of active ingredient, in this case salicylic acid, calculated to produce the desired effect in association with the selected formulation. Preferred dosages of aspirin for a variety of therapeutic and prophylactic purposes are well known in the art. Appropriate dosages of gaultherin, which comprises the same active ingredient as aspirin, may be easily determined by standard methods.

The following example is provided to describe the invention in greater detail. It is intended to illustrate, not to limit, the invention.

### EXAMPLE I

### Preparation of Gaultherin from Gaultheria procumbens

Wintergreen (Gaultheria procumbens) is a small ericaceous plant found growing in the understory of dense forests and is also widely used in the landscape industry. Wintergreen is known for its constituent essential oil of wintergreen which is comprised predominantly of methyl salicylate (Tyler et al., 1981). In this example, the salicylate concentrations of several plant species are compared, and it is shown that wintergreen contains extraordinarily high concentrations of salicylates, which can reach concentrations of over 1% of the fresh weight of the tissue. The predominant form of this salicylate is gaultherin. Methods for obtaining gaultherin from Gaultheria procumbens are described.

METHODS

Measurement of SA using stable isotope dilution analysis. Plant tissue or extract samples (100-1000 mg) were ground in liquid nitrogen and extracted in 5 mL of 90% methanol as previously described (Ribnicky et al., 1998). Samples which were not subjected to immediate analysis were stored at −80° C. ²H₆-SA (98% atom ²H6 enriched, Isotec Inc., Miamisburg, Ohio) dissolved in isopropanol was added to each sample as an internal standard in the amount of 0.5-1 μg, depending on the size of the sample and expected concentration of salicylate. The sample was extracted at 4° C. for 1 h in a 13×100 mm test tube and centrifuged at 10,000g for 10 min. The pellet was then rinsed with 1 mL 100% methanol followed by an additional centrifugation. The samples to be analyzed for free SA were processed in the same manner as for the free acids previously described (Ribnicky et al., 1998). The free SA samples of wintergreen were processed using an alternative method as well to prevent any possible liberation of free SA from methyl salicylate. These samples were extracted in 50% isopropanol/100 mM phosphate buffer, pH 7 at 4° C. for 1 h and diluted 10 fold with water. The sample was then partially purified on a 3 mL conditioned (rinsed with 2 mL of each methanol, water and 100 mM phosphate buffer, pH 7, followed by a rinse with 8 mL water) amino solid phase extraction column (J. T. Baker, Phillipsburg, N.J.). The sample was then eluted with 4% acetic acid/methanol, dried in vacuo followed by derivatization and analysis as described above. The samples to be analyzed for total acids were also processed as previously described (Ribnicky et al., 1998, Enyedi et al., 1992), with modifications. The methanolic extract was resuspended in 2 mL of 2N NaOH and sealed in a 12×35 mm screw cap vial with a TFE lined cap prior to heating at 70° C. After 2 h, the sample was cooled on ice to 4° C. and then it was acidified with 250 μL of 36.9% HCl The sample was then resealed and heated for an additional 1 h at 70° C., cooled to 4° C. and partitioned twice with ethyl acetate:cyclopentane:2-propanol (100:99:1) and reduced in vacuo to dryness. After resuspending in 2 mL of 1% acetic acid, the sample was applied to a conditioned (rinsed with 2 mL each water, methanol and 0.5% acetic acid) C₁₈ SPE column (J. T. Baker), rinsed with 5 mL of water and eluted with 2 mL of acetonitrile, reduced to dryness and methylated with ethereal diazomethane. The methylated sample was reduced to near dryness and resuspended in 25 μL of ethyl acetate for analysis by GC-MS-selected ion monitoring. The samples were manually injected in the splitless mode into a gas chromatograph (model 5890, Hewlett-Packard) /mass spectrometer (model 5971, Hewlett-Packard) equipped with a 30-m×0.25 mm DB-5MS fused silica capillary column (J&W Scientific, Folsom Calif.). Chromatographic parameters were as follows: injection temperature at 280° C., initial oven temperature at 50° C. for 3 min followed by a ramp at 30° C./ min to 280° C. The monitored ions for native methyl salicylate were (m/z): 92, 120 and 152 and for the ²H₆-methyl salicylate were 96, 124 and 156. The concentration of the endogenous SA was then calculated based on the ratio of the major ion (120) of the native SA and the comparable ion (124) of the ²H₆-labeled internal standard using the equation of isotope dilution analysis described by Cohen et al., 1986. These ratios were also confirmed using the abundance of the molecular ions of both forms (152 and 156). The decrease in mass from 46 to 44 for the deuterium-labeled internal standard is due to the exchange of the carboxyl and hydroxyl hydrogens of the molecule. The other 4 positions are non-exchangeable.

Measurement of methyl salicylate using stable isotope dilution analysis. Measurement of endogenous methyl salicylate was performed as previously described for benzaldehyde (Ribnicky et al., 1998) ²H₄-methyl salicylate was added during the extraction for use as an internal standard. The ²H₄-methyl salicylate was custom synthesized for this purpose. Five hundred μg of ²H₆-SA dissolved in isopropanol was dried under a stream of nitrogen gas in a 12×35 mm screw cap vial and resuspended in 100 μL of methanol. One mL of ethereal diazomethane was then added to the labeled SA solution and the vial was sealed with a TFE lined cap. After 10 min, this mixture was reduced to near dryness with a stream of nitrogen gas and resuspended in 10 mL of isopropanol. The precise concentration of the ²H₄-methyl salicylate solution was adjusted to account for the recovery of the chemical synthesis. The degree of adjustment was small and was determined by mixing the ²H₄-labeled internal standard with known amounts of unlabeled methyl salicylate and comparing the abundance of the major ions of both molecular forms by GC-MS as described above for SA.

HPLC and LC-MS analysis of gaultherin. In order to extract the conjugates of salicylate, fresh plant tissues or tissues which were stored frozen at −20° C. or lower, were frozen in liquid nitrogen and ground with a mortar and pestle. Tissues which were dried at 20° C. or 70° C. or freeze dried did not contain measurable concentrations of conjugate or methyl salicylate. Alcohol was then added to the tissue while it remained frozen in a volume at least 5-fold greater than the corresponding weight of the tissue. Alternatively, for larger extractions, fresh tissue was ground in a Waring blender at high speed in the presence of alcohol until the tissue became a suspension of fine particulates. Solid plant materials were then removed by centrifugation after a minimum incubation of 1 h at 4° C. as described for the measurements of salicylates. Extraction of wintergreen in alcohols, including methanol, ethanol or isopropanol, yields one predominant component as determined by HPLC (Waters) equipped with a Waters 996 photodiode array detector (PDA)(scan from 190-450 nm) and a Waters 474 scanning fluorescence detector (set at an excitation wavelength of 301 nm and an emission of 412 nm) using a C₁₈ column (Waters)(4.6mm×15Omm). The column was equilibrated at a flow of 1.5 mL/min with 0.5% acetic acid:methanol (75:25 v/v) followed by a gradient to 25:65 starting at 3 min and ending at 10 min. The primary metabolite eluted at 5 min using these conditions. This metabolite was absent from water extracted tissues which instead contained peaks with retention times and PDA spectra corresponding to salicylic acid and methyl salicylate. This conjugated form of methyl salicylate was relatively polar but was better resolved with reverse phase HPLC using a shallow gradient from 5:95 to 25:75 (acetonitrile: 0.5% acetic acid in water) over 30 min with a C₈ column (4.6 mm×150 mm) and a flow rate of 1 mL/min. These conditions were used for the purification of the salicylate conjugate (by collecting the fraction at 17 min) and revealed the presence of a second form of bound salicylate (which eluted at 10 min). The minor form of bound salicylate which eluted early was not always detectable. Further characterization of the salicylate forms were performed by LC-MS as well with a Waters Integrity System equipped with a 996 photodiode array detector and Thermabeam Mass Detector. A Waters semi-microbore Nova Pak C8 column (2mmXl5Omm) was equilibrated with 0.5% acetic acid:acetonitrile (95:5, v/v) with a flow of 0.25 mL/min. After injection, a gradient to a final solvent composition of 5:95, v/v, was established over 25 min. The solvent composition was then returned to initial over 2 min and equilibrated for 15 min prior to subsequent injections. With the LC-MS conditions, the retention time of the major metabolite was 12 min and the minor metabolite (not always detectable) was 3 min. The Thermabeam Mass Detector operates with standard electron impact ionization energy of 70 eV and scans from 50 to 700 m/z. The fragmentation pattern of the conjugated forms, as seen in the lower mass ranges (50 - 100 m/z), suggested the presence of a sugar within the larger molecule. This conclusion was reached by matching these spectra with the Wiley Registry of Mass Spectral Data. Many sugars, however, are characterized by similar fragmentation patterns. Preliminary analysis of the dried wintergreen extract using β-glucosidase, which is specific for glucose conjugates, showed a 6-fold increase in the release of free salicylate as compared to a 330-fold increase in the release of free salicylate following complete chemical hydrolysis (Enyedi et al., 1992). A similar trend was observed for the release of methyl salicylate from this extract. Together, these results suggest that the predominant form of salicylate in wintergreen leaves is not a simple glucose conjugate but does contain sugar. Early reports describe gaultherin as a conjugate of methyl salicylate to a disaccharide of xylose and glucose. In order to confirm these observations, an HPLC purified fraction of the major peak was hydrolyzed in 0.5% HCl at 80° C. for 3 h followed by acetylation at 60° C. for 12 h with 1 mL of chloroform, 200 mL of acetic anhydride (Supelco, Bellafont Pa.) and 400 mL of glacial acetic acid. Similar acetylations were performed on equimolar mixes of sugars. Only the combination of xylose and glucose showed the same GC-MS profile as the hydrolysis products from the conjugate, thus confirming the identity reported in the literature. In addition, positive fast atomic bombardment (FAB+) analysis of the purified conjugate revealed a molecular ion of 469 m/z. This mass corresponds to the mass of gaultherin (446 m/z) plus a molecule of sodium, which is frequently present in samples.

RESULTS

The concentrations of free and total SA in various species were measured using stable isotope dilution analysis as described above. Results are shown in Table I.

TABLE 1
The amounts of free and total SA in species
reported to contain elevated concentrations of SA as
measured by stable isotope dilution analysis with GC-MS

| | Free SA | Total SA |
|---|---|---|
| Plant Species | μg/g FW | μg/g FW |
| English thyme | 0.81 | 31.63 |
| Lemon thyme | 1.55 | 42.32 |
| French thyme | 0.33 | 13.26 |
| Lavender | 0.28 | 6.14 |
| Rosemary | 0.58 | 3.84 |
| Rice M-201 | 1.40 | 9.50 |
| Wintergreen | 19.00 | 5770.00 |

These plants represent just a few of the species examined which were suggested to contain potentially elevated concentrations of SA as suggested by various literature sources (Perry et al., 1996, Raskin et al., 1990) and as such could represent alternative sources of natural aspirin. The method of stable isotope dilution analysis provided both precise quantification of SA and chemical identification, independent of sample recovery. A similar method using an unpurified ²H₃-SA has been described but was limited to the measurement of only free SA (Scott and Yamamoto, 1994). The many advantages of stable isotope dilution analysis are critical when examining a diverse variety of plant species. Clearly wintergreen contains the highest concentration of SA, which is expected of a plant which is know for the presence of the oil of wintergreen, an essential oil consisting primarily of methyl salicylate. The SA concentrations of the other species were generally much lower than reported in the literature which utilized less precise analytical methods.

No documented varieties or cultivars of wintergreen, Gaultheria procumbens, have been described in the literature to our knowledge. However, there were clearly differences in appearance in those plants which were grown on the west coast from those plants which were grown on the east coast. In general, the west coast plants were anatomically larger in height, leaf size, flower size and berry size. The SA contents of these varieties was examined to determine if there was any correlation between size and salicylate concentration.

TABLE II
Salicylate content of wintergreen varieties

| | West Coast | East Coast |
|---|---|---|
| Salicylate pool | μg/g FW | μg/g FW |
| Methyl salicylate | 85 | 106 |
| Free salicylic acid | 1,661 | 19 |
| Total salicylate | 10,700 | 5,770 |

Table II shows that the larger variety contains almost twice as much total salicylate per gram fresh weight of tissue. In both varieties, however, the methyl salicylate concentrations were similar and several-fold lower than the total salicylate concentration. Therefore, the bulk of the methyl salicylate must be present in a conjugated form, i.e., gaultherin. The difference in the size of the plants grown on the west coast from those plants grown on the east coast could be attributed to different growing conditions or to slight varietal differences. This distinction cannot yet be made. We have observed, however, that the growing conditions ofGaultheria procumbens can have a dramatic effect on the concentrations of salicylate within them. In general, stress tends to promote elevated concentrations.

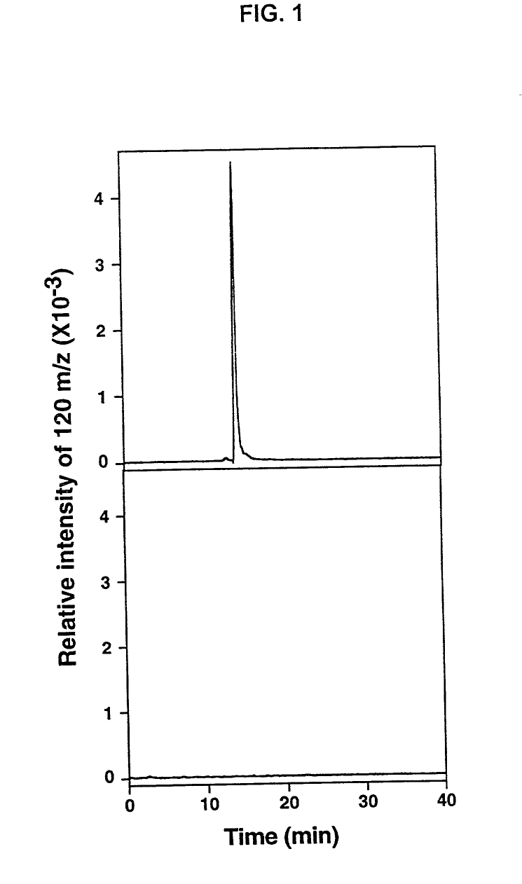

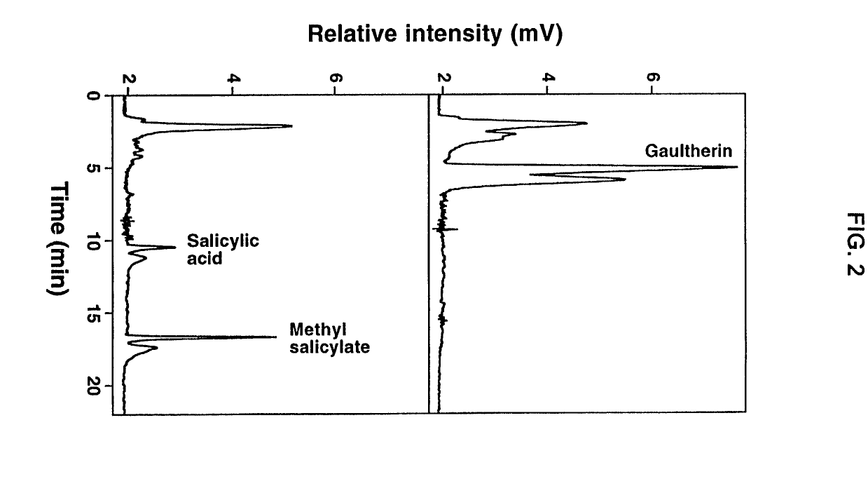

Two different kinds of extractions were performed on wintergreen leaf tissue in order to determine the form of the SA in the leaf tissue since the methyl salicylate and free SA concentrations were relatively low compared to the total SA concentration (FIG. 1 and FIG. 2). The HPLC fluorescence profile of water extracted leaves contained only 2 predominant peaks (in addition to the solvent front) which had retention times and PDA spectra corresponding to SA and free methyl salicylate (FIG. 1). The water extracted sample contains potential products of enzymatic activity after tissue disruption. Methanolic extracts of wintergreen leaves had only one predominant peak (in addition to the solvent front, FIG. 1) which had a very early retention time compared to SA and methyl salicylate. Only very small peaks representing SA and methyl salicylate were observed in the methanolic extracts. The methanol denatures proteins and prevents degradation of metabolites from enzymatic activity. The dominant presence of one peak with fluorescence suggested that it would consist of a bound form of SA. These extraction results predict a high concentration of bound SA was present in the wintergreen tissue which was protected from enzymatic activity and that this form was degraded in enzymatically active tissues upon damage.

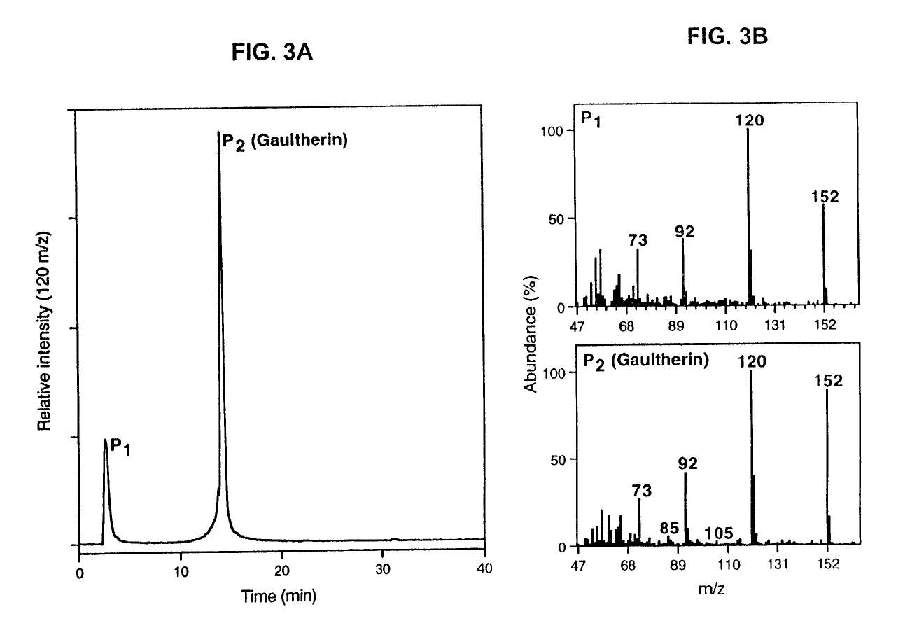

The unknown which was presumed to be a bound form of SA was further characterized by LC-MS. The use of a shallow gradient and semi-microbore column was able to extend the retention time of this metabolite and provide enhanced chromatography as shown in FIG. 3. This bound form of SA was also resolved into 2 forms, abbreviated P1 and P2. P1 was a minor form which was not always present in alcoholic extracts but could represent an important additional metabolite. Both P1 and P2 were characterized by similar mass spectra. The most noteworthy feature of these spectra is that they consist primarily of the fragmentation pattern of methyl salicylate. The molecule of methyl salicylate would never be visible by LC-MS as methyl salicylate is a volatile molecule which would be lost during desolvation, prior to MS ionization. The loss of volatile analytes during desolvation has been extensively observed. The occurrence of a molecule which contains the ion fragments associated with methyl salicylate by LC-MS would result only from the fragmentation of a molecule which contains methyl salicylate as part of a larger chemical structure with lower volatility. Many of the smaller ions of these spectra (less than 92) are not characteristic of the fragmentation pattern of methyl salicylate and are likely to represent ions from the non-methyl salicylate portion of the larger molecule. In general, these smaller ions are typical fragments from sugars, as suggested by comparison between those of the Wiley Registry of Mass Spectral Data. Many simple sugars possess similar spectra which prevents the determination of their precise structure from this data alone. The fragmentation pattern of these forms by LC-MS is, therefore, consistent with a sugar conjugate of methyl salicylate. No molecular ion seems to be present in these spectra which would also be indicative of sugar conjugates that are commonly unable to survive electron impact ionization without fragmentation.

Various species were investigated for comparative purposes and as potential new sources of natural aspirin as shown in Table III.

TABLE III
Gaultherin and salicylate content of select species

| | Gaultherin | Total Salicylate |
|---|---|---|
| Plant Species | μg/g FW | μg/g FW |
| Gaultheria procumbens | 26,000* | 10,700 |
| Gaultheria shallon | n.d. | 1.2 |
| Filipendula ulmaria | n.d. | 479 |

*based on extract concentrations
n.d. = not detectable

Gaultheria shallon is a close relative of Gaultheria procumbens but has the advantage of much greater biomass. Gaultheria shallon does not contain measurable concentrations of gaultherin and has very small concentrations of total salicylate as well. Filipendula ulmaria is not a related species but has been reported in the literature to be the original source of natural aspirin used by ancient peoples (Balick and Cox, 1997). This contrasts other literature which portrays willow as the original source of aspirin (Pierpoint, 1994). Filipendula ulmaria does contain elevated concentrations of total salicylate, as compared to those in Table I, albeit much lower than those found in Gaultheria procumbens. Filipendula does not, however, contain measurable concentrations of gaultherin. Only the leaves of this species were examined, not the roots or the berries which may have additional forms. The concentrations within the leaves were very low when compared to Gaultheria procumbens and did not warrant further investigation.

Gaultheria procumbens is the richest source of total salicylate as determined using our methods of stable isotope dilution with the plant species mentioned above. All of the previous investigations were performed with leaf tissue only, which may not be the only tissues which contain high concentrations of salicylate. Use of the entire plant above the ground, however, would be ideal for practical reasons of harvest. Therefore, each of the tissues was examined for total salicylates content as shown in Table IV.

TABLE IV
Total salicylate content of wintergreen tissues

| Tissue | SA (μg/g FW) |
|---|---|
| Leaves | 3,832 |
| Flowers | 6,425 |
| Stems | 2,227 |
| Berries | 1,491 |

All of the tissues examined contained substantial concentrations of salicylate as compared to other species (Table I & Table IV), the flowers being the organ which seems to contain the highest concentration. The specific form of salicylates within each tissue was not determined. The results from the analysis of the leaves suggest that the gaultherin conjugate would be the most prevalent form.

Further measurements were performed with alcoholic extracts of Gaultheria procumbens. A variety of alcohols were used for the efficient extraction of gaultherin which include methanol, ethanol and isopropanol. The concentration of total salicylate within these dried extracts ranged from 37 to 60 mg/g (average of 5%) as measured using stable isotope dilution analysis by GC-MS. This corresponds to a gaultherin concentration range of 120-180 mg/g (average of 15%) as measured by HPLC and LC-MS. Based on the difference in mass of salicylic acid and gaultherin, which is 3.2 times, these two ranges agree closely. Therefore, not only do these samples contain high concentrations of total salicylate and gaultherin, but the close agreement of these concentrations predicts that most of the salicylate present must be in the form of gaultherin.

In this example, the identity of gaultherin was verified using modern analytical methods including GC-MS and LC-MS. Methods were also defined for the precise quantification of SA, methyl salicylate and total salicylate using stable isotope dilution technologies. A systematic measurement of the individual salicylates in wintergreen has not previously been performed. These methods have been used to measure salicylate concentrations in various species and determine that Gaultheria procumbens is the richest plant source of gaultherin.

The predominant form of salicylate in Gaultheria procumbens is gaultherin, which is easily hydrolyzed upon tissue disruption. This hydrolysis can be prevented, however, with proper extraction in alcohol or other solvent, which presumably inactivates the activity of gaultherase and leads to the production of extracts containing as much as 18% gaultherin.

REFERENCES

Balick MJ, Cox PA (1997) Plants that heal. In MJ Balick and PA Cox eds, Plants, People and Culture. Scientific American Library. New York, p 32.

Barnett HJM, Hirsh J, Mustard JF eds. (1982) In Acetylsalicylic Acid, New Uses for and Old Drug. Raven Press, New York

Bridel MM, Grillon S (1928) Le glucoside a salicylate de methyle du Gaultheria procumbens L. este le monotropitoside. Comptes Rendus pp 609-611

Bridel MM, Guignard ML (1923)a Etude biochimique sur la composition du Monotropa hypopitys L. Obtention d un nouveau glucoside a salicylate de methyle, la monotropitine. Comptes Rendus pp 642-644

Bridel MM, Guignard ML (1923)b Sur 1 hydrolyse fermentaire de la monotropitine. Obtention du primeverose. Comptes Rendus pp 991-993

Bridel MM, Guignard ML (1925)a Sur un complexe glucosidique instable de 1 ecorce de tige de nerpun purgati (Rhamnus cathartica L.). Comptes Rendus pp 857-860

Bridel MM, Guignard ML (1925)b Le primeverose, les primeveroides et la primeverosidase. Comptes Rendus pp 1421-1423 Bridel MM, Picard P, Guignard ML (1925) Sur la preparation et les propietes du monotropitoside. Comptes Rendus pp 1864-1866

Enyedi AJ, Yalpani N, Silverman P, Raskin I (1992) Localization, conjugation, and function of salicylic acid in tobacco during the hypersensitive reaction to tobacco mosaic virus. Proc Natl Acad Sci 89:2480-2484

Goris A, Vischniac C, Guignard ML (1919) Caracteres et composition du primeverose. Comptes Rendus pp 871-873

Perry CA, Dwyer J, Gelfand JA, Couris R, McClosky WW (1996) Health effects of salicylates in food and drugs. Nutrition Reviews 54:225-240

Pierpoint, WS Salicylic acid and its derivatives in plants: medicines, metabolites and messenger molecules (1994) In Advances in Botanical Research, Vol. 20 pp 164-235

Proctor W (1844) Observation on the volatile oil of Betula lenta, and on gaultherin, a substance which, by its composition, yields that oil. American Journal of Pharmacy 9:241-250

Raskin I, Skubatz H, Tang W, Meeuse BJD (1990) Salicylic acid in thermogenic and non-thermogenic plants. Annals of Botany 66:369-370

Robertson A, Walters RB (1931) Synthesis of glucosides. part VII, the synthesis of monotropidoside (gaultherin). Journal of the American Chemical Society. pp 1881-1888

Scott IM, Yamamoto H (1994) Mass spectrometric quantification of salicylic acid in plant tissues Phytochemistry 37:335-336

Tyler VE, Brady LR, Robbers JE (1981) Volatile oils. In VE Tyler, LR Brady LE Robbers eds. Pharmacognasy. Lea & Febiger, Philadelphia, pplO3-143

Wiley Registry of Mass Spectral Data (1994), ⁶th Ed. with structures; John Wiley & Sons, Inc.

The present invention is not limited to the embodiments described and exemplified above, but is capable of variation and modification without departure from the scope of the appended claims.

We claim:

1. An extract of plant tissue comprising at least 5 mg gaultherin per gram fresh weight of plant tissue, wherein said extract is obtained by disrupting said plant tissue under solvent conditions lacking a drying agent.

2. The extract of claim 1, comprising at least 15 mg gaultherin per gram fresh weight plant tissue.

3. The extract of claim 1, comprising at least 25 mg gaultherin per gram fresh weight plant tissue.

4. The extract of claim 1, prepared from Gaultheria procumbens.

5. The extract of claim 1, which is an alcoholic extract.

6. The extract of claim 5, wherein the alcohol is selected from the group consisting of methanol, ethanol and isopropanol.

7. The extract of claim 6, wherein the alcohol is ethanol.

8. A powdered preparation of gaultherin obtained from the extract of claim 1.

9. An aqueous solution of gaultherin obtained from the powdered preparation of claim 8.

10. A method of obtaining gaultherin from plant tissue, in an amount of at least 5 mg per gram fresh weight of the plant tissue, which comprises:
(a) providing fresh or fresh-frozen plant tissue; and
(b) preparing an extract of the tissue by disrupting the tissue under conditions lacking a drying agent that reduce the likelihood of hydrolysis of the gaultherin, thereby producing an extract of the plant tissue comprising the at least 5 mg gaultherin per gram fresh weight of the plant tissue.

11. The method of claim 10, wherein the solvent is an alcohol selected from the group consisting of methanol, ethanol and isopropanol.

12. The method of claim 11, wherein the solution comprises the alcohol in an amount of at least 30% by volume.

13. The method of claim 11, wherein the solvent is ethanol.

14. The method of claim 10, wherein the plant tissue is obtained from Gaultheria procumbens.

15. The method of claim 14, wherein the plant tissue comprises above-ground plant parts.

16. The method of claim 10, wherein the plants are subjected to an environmental stress prior to harvesting the plant tissue.

17. The method of claim 16, wherein the environmental stress is selected from the group consisting of heat stress, drought stress and stress resulting from treatment with chemical elicitors.

18. The method of claim 10, which further comprises freezing the plant tissue in liquid nitrogen before disrupting it.

19. The method of claim 10, which further comprises removing solid plant material from the extract.

20. The method of claim 19, which further comprises removing solvent from the extract.

21. The method of claim 20, which further comprises adding water to the dried extract, thereby producing an aqueous solution of gaultherin.

22. The method of claim 21, which further comprises separating water-insoluble components from the aqueous solution and removing the aqueous solvent to produce a dried preparation of gaultherin.

23. An extract comprising at least 5 mg gaultherin per gram fresh weight plant tissue, prepared by the method of claim 10.

24. A method of obtaining gaultherin from Gaultheria procumbens, which comprises:
a) providing fresh or fresh-frozen Gaultheria procumbens plant material; and
b) disrupting the tissue in a solution comprising at least 30% by volume of alcohol under solvent conditions lacking a drying agent, thereby producing an alcoholic extract of the tissue comprising the gaultherin.
